(19) United States
(12) Patent Application Publication
(10) Pub. No.: US 2022/0305177 A1
(43) Pub. Date: Sep. 29, 2022

(54) THREAD AND METHOD FOR PREPARING SAME

(71) Applicant: National Agriculture and Food Research Organization, Tsukuba-shi, Ibaraki (JP)

(72) Inventor: Toshiaki Takezawa, Tsukuba-shi, Ibaraki (JP)

(21) Appl. No.: 17/594,944

(22) PCT Filed: Apr. 30, 2020
(86) PCT No.: PCT/JP2020/018235

§ 371 Date: Nov. 3, 2021

(30) Foreign Application Priority Data
May 13, 2019 (JP) 2019-090900

(51) Int. Cl. A61L 27/52; D02G 3/10; A61L 2/00; A61L 17/10; A61L 17/14; A61L 17/08

(52) U.S. Cl. CPC A61L 27/52; D02G 3/10; A61L 2/0047; A61L 17/10; A61L 17/14; A61L 17/08

(57) ABSTRACT

This method for preparing a thread has a step for obtaining a thread-shaped vitrigel by wetting a first sheet-shaped hydrogel dried body or a sheet-shaped vitrigel dried body in a first aqueous solution and twisting the same into a thread shape.

### TECHNICAL FIELD

The present invention relates to a thread and a method for preparing the same.

Priority is claimed on Japanese Patent Application No. 2019-90900, filed May 13, 2019, the content of which is incorporated herein by reference.

### BACKGROUND ART

In the field of regenerative medicine, there is an urgent need for technological development to form a framework of tissues and organs by knitting thread-shaped material which functions as cell scaffolds.

The inventor of the present invention has so far developed a technique for processing columnar-shaped native collagen gel into a thread shape and then processing columnar-shaped atelocollagen gel irradiated with UV into a thread shape with the aim of practical use as a tissue regenerating thread (a suture thread, an indwelling thread, a cell transplantation thread, or the like) in the field of regenerative medicine (for example, refer to Patent Literature 1 and 2)

### CITATION LIST

### Patent Literature

[Patent Literature 1]
Japanese Patent No. 4677559

[Patent Literature 2]
PCT International Publication No. WO 2018/211877

### SUMMARY OF INVENTION

### Technical Problem

The inventor of the present invention has aimed to develop a thread-shaped atelocollagen vitrigel having superior practicality on the basis of the development results described above.

The present invention was made in view of the above circumstances and provides an easy technique for preparing a thread-shaped atelocollagen vitrigel having an infinite length and a dried body thereof.

### Solution to Problem

The present invention includes the following aspects:

[1] A method for preparing a thread including: a step for obtaining a thread-shaped vitrigel by wetting a first sheet-shaped hydrogel dried body or a sheet-shaped vitrigel dried body in a first aqueous solution and twisting the same into a thread shape.
[2] The method for preparing a thread according to [1] comprising:
a step for obtaining a thread-shaped vitrigel by overlapping an end portion, which is left without threading, of the first sheet-shaped hydrogel dried body or the sheet-shaped vitrigel dried body and an end portion of a second sheet-shaped hydrogel dried body or a sheet-shaped vitrigel dried body to form a joint portion and wetting the joint portion and the second sheet-shaped hydrogel dried body or the sheet-shaped vitrigel dried body in the first aqueous solution and twisting the same into a thread shape.
[3] The method for preparing a thread according to [1] or [2] including: a step for obtaining a thread-shaped vitrigel dried body by drying the thread-shaped vitrigel.

[4] The method for preparing a thread according to [3] including: a step for wetting the thread-shaped vitrigel dried body in a second aqueous solution and drying the same.

[5] The method for preparing a thread according to [3] or [4] including: a step for irradiating the thread-shaped vitrigel dried body with ultraviolet light.

[6] The method for preparing a thread according to any one of [1] to [5], wherein the hydrogel is atelocollagen gel.
[7] The method for preparing a thread according to any one of [1] to [6], wherein the first aqueous solution is water or an atelocollagen sol.

[8] The method for preparing a thread according to any one of [2] to [6], wherein the end portion is a protrusion portion.

[9] The method for preparing a thread according to any one of [4] to [8], wherein the second aqueous solution is an atelocollagen sol.

[10] The method for preparing a thread according to any one of [1] to [9], including: a step for obtaining a sheet-shaped hydrogel by injecting a sol into a mold, gelling the sol, and then removing the mold; and a step for obtaining a sheet-shaped hydrogel dried body by drying and vitrifying the sheet-shaped hydrogel.

[11] The method for preparing a thread according to [10] including: a step for irradiating the sheet-shaped hydrogel dried body with ultraviolet rays.

[12] The method for preparing a thread according to [10] or [11] including: a step for obtaining a sheet-shaped vitrigel by rehydrating the sheet-shaped hydrogel dried body or the sheet-shaped hydrogel dried body irradiated with ultraviolet rays; and a step for obtaining a sheet-shaped vitrigel dried body by drying and revitrifying the sheet-shaped vitrigel.

[13] The method for preparing a thread according to [12] including: a step for irradiating the sheet-shaped vitrigel dried body with ultraviolet rays.

[14] A thread including: a vitrigel dried body; and having elasticity at the time of hydration.

[15] The thread according to [14], which has a spiral structure.

[16] The thread according to [14] or [15], wherein the vitrigel dried body is an atelocollagen vitrigel dried body.

### Advantageous Effects of Invention

According to the present invention, it is possible to provide an easy technique for preparing a thread-shaped atelocollagen vitrigel having an infinite length and a dried body thereof.

### BRIEF DESCRIPTION OF DRAWINGS

FIG. 1 is an example of a sheet-shaped vitrigel dried body used in an embodiment.

FIG. 2 is an example of a step for preparing the sheet-shaped vitrigel dried body used in the embodiment.

FIG. 3 is an example of a method for preparing a thread in an embodiment.

FIG. 4 is an example of the method for preparing a thread in the embodiment.

FIG. 5 is a photograph of a thread prepared in Preparation Example 1.

FIG. 6 is a test result in Example 1.

FIG. 7 is a test result in Example 1.

FIG. 8 is a test result in Example 1.

FIG. 9 is a test result in Example 1.

### DESCRIPTION OF EMBODIMENTS

Embodiments of the present invention will be described in detail below with reference to the drawings in some cases. The dimensional ratios in the drawings may be exaggerated for explanation and the dimensional ratios in each of the drawings may not necessarily match actual dimensional ratios.

<<Method for preparing thread>>

In an embodiment, the present invention provides a method for preparing a thread which has a step E for obtaining a thread-shaped vitrigel by wetting a first sheet-shaped hydrogel dried body or a sheet-shaped vitrigel dried body in a first aqueous solution and twisting the same into a thread shape.

First, the method for preparing a sheet-shaped hydrogel dried body will be described.

The method for preparing a sheet-shaped hydrogel dried body has a step A for obtaining a sheet-shaped hydrogel by injecting a sol into a mold, gelling the sol, and then removing the mold and a step B for obtaining a sheet-shaped hydrogel dried body by drying and vitrifying the sheet-shaped hydrogel.

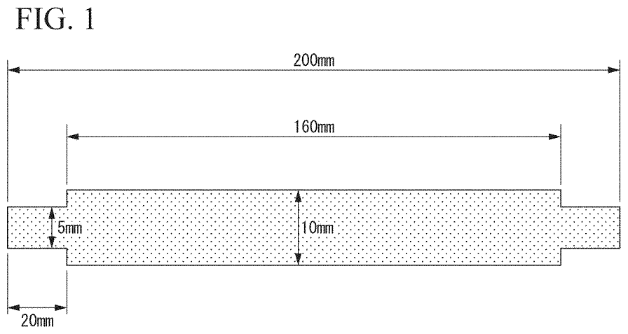

FIG. 1 shows an example of the sheet-shaped hydrogel dried body. As shown in FIG. 1, it is preferable that an end portion of the sheet-shaped hydrogel dried body has a protrusion portion for ease of twisting. Furthermore, in FIG. 1, the sheet-shaped hydrogel dried body has a strip shape and a narrow width, but the width is not limited thereto and can be appropriately adjusted. When a width of the sheet-shaped hydrogel dried body is large, a thicker thread is provided and when the width of the sheet-shaped hydrogel dried body is small, a thinner thread is provided. In this way, when the width of the sheet-shaped hydrogel dried body is controlled, it is possible to adjust a thickness of the thread.

Also, the width of the sheet-shaped hydrogel dried body does not need to be uniform, lengths of widths may be different as appropriate such as in a gourd type, and lengths of widths may gradually become different such as in a triangular shape. Furthermore, the sheet-shaped hydrogel dried body may be a perforated, for example, may be a porous sheet-shaped hydrogel dried body.

[Step A]

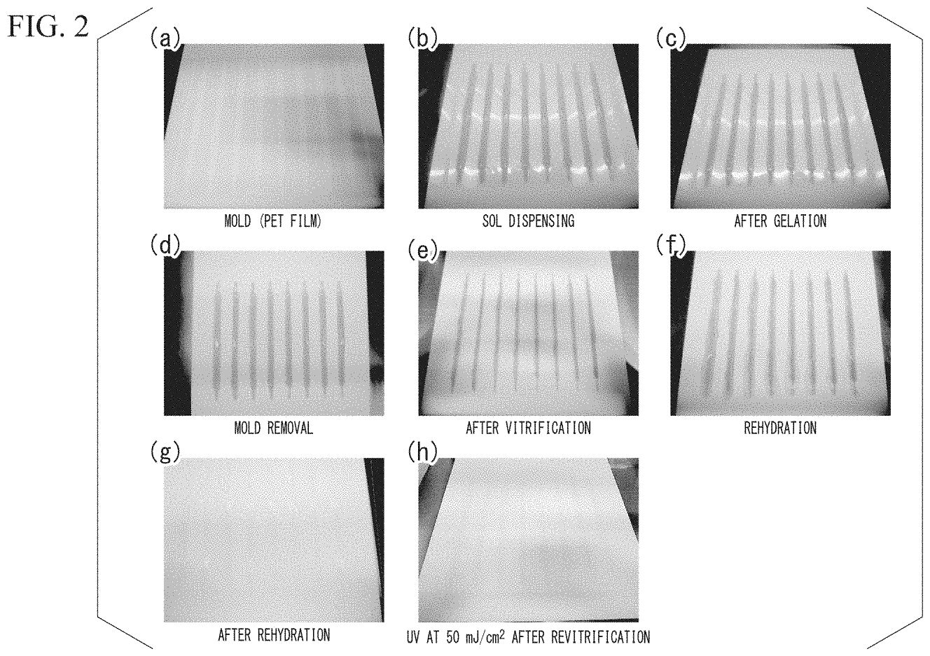

Step A is a step for obtaining a sheet-shaped hydrogel by injecting sol into a mold, gelling the sol, and then removing the mold (refer to (a) to (d) of FIG. 2).

The mold is not particularly limited as long as a desired sheet-shaped hydrogel shape is hollowed out from the mold and examples of the mold include a PET film in which a plurality of sheet-shaped hydrogel shapes are hollowed out as shown in (a) of FIG. 2.

In this specification, the term “sol” means dispersoid colloidal particles (size: about 1 to several hundred nm) particularly composed of a polymer compound in which a liquid is used as a dispersion medium. More specific examples of the sol include aqueous solutions such as natural polymer compounds and synthetic polymer compounds. When these polymer compounds have a network structure having cross-linking introduced through chemical bonds, the polymer compounds transition to “hydrogels” which are semi-solid state substances which hold a large amount of water in a network thereof. That is to say, a “hydrogel” refers to a hydrogel obtained by gelling a sol.

The sol used as a raw material for the sheet-shaped hydrogel may be any sol as long as the sol is a material having biocompatibility. In addition, examples of the sol include natural polymer compounds such as gelling extracellular matrix-derived components, fibrin, agar, agarose, and cellulose, and synthetic polymer compounds such as polyacrylamide, polyvinyl alcohol, polyethylene oxide, and poly(II-hydroxyethylmethyllate/polycaprolactone.

Examples of the gelling extracellular matrix-derived components include collagen (type I, type IQ, type III, type V, type XI, and the like), basement membrane components (trade name: Matrigel) reconstituted from a mouse EHS tumor extract (type IV collagen, laminin, heparan sulfate proteoglycan, or the like), glycosaminoglycan, hyaluroic acid, proteoglycan, gelatin, and the like, but the present invention is not limited thereto. It is possible to prepare a desired sheet-shaped hydrogel by selecting components such as salts, and their concentrations, pH, and the like most suitable for each gelation. In addition, when these raw materials are combined, it is possible to obtain a sheet-shaped hydrogel which imitates various in-vivo tissues.

Among these, as the sol, a gelling extracellular matrix-derived component is preferable and collagen is more preferable. Furthermore, as a more preferable raw material among collagens, native collagen, or atelocollagen can be exemplified and atelocollagen is more preferable.

The “vitrigel” refers to a gel in a stable state obtained by performing vitrification on a hydrogel in the related art and then rehydrating the vitrificated hydrogel and has been named “vitrigel (registered trademark)” by the inventor of the present invention.

Also, in this specification, when the preparing step in the embodiment is described in detail, a dried body of a hydrogel immediately after the vitrification step and not undergoing the rehydration step is simply referred to as a “hydrogel dried body.” Moreover, the gel obtained through the rehydration step after the vitrification step is distinguished and represented as “vitrigel” and the dried body obtained by vitrifying the vitrigel is referred to as a “vitrigel dried body.” Furthermore, the body obtained by subjecting the vitrigel dried body to the step for performing irradiation with ultraviolet rays is referred to as a “vitrigel dried body irradiated with ultraviolet rays.” Therefore, the “vitrigel” is a hydrate.

Furthermore, in this specification, when the term “vitrigel” is used, the term “(registered trademark)” may be omitted in some cases.

In Step A, when the sol is injected into the mold, if an amount of the sol is increased, it is possible to obtain a thick sheet-shaped hydrogel, and as a result, it is possible to obtain a thick thread. Furthermore, if an amount of the sol is reduced, it is possible to obtain a thin sheet-shaped hydrogel, and as a result, it is possible to obtain a thin thread. In this way, when an amount of the sol which is injected is adjusted, it is possible to control a thickness of a thread.

A thickness of the sheet-shaped hydrogel is preferably 0.1 mm to 20 mm, more preferably 0.5 mm to 4 mm, and still more preferably 1 mm to 2 mm.

In Step A, a temperature at which the sol is kept warm during gelation may be appropriately adjusted in accordance with a type of the sol to be used. For example, when the sol is a collagen sol, a temperature of the heat retention at the time of gelation may be any heat retention temperature as long as the heat retention temperature is lower than a denaturation temperature of a collagen depending on an animal species of a collagen to be used. In addition, generally, it is possible to perform gelation in a few minutes to a few hours by keeping a temperature at 20° C. or higher and 37° C. or lower.

[Step B]

Step B is a step for obtaining a sheet-shaped hydrogel dried body by drying and vitrifying the sheet-shaped hydrogel (refer to (e) of FIG. 2). When the sheet-shaped hydrogel is dried, it is possible to fully remove the free water in the sheet-shaped hydrogel and further cause partial removal of bound water to proceed.

As a time period of this vitrification step (the step for fully removing the free water in the sheet-shaped hydrogel and then causing partial removal of the bound water to proceed) is increased, when rehydration is performed, it is possible to obtain a sheet-shaped vitrigel having excellent transparency and strength. It is possible to wash the sheet-shaped vitrigel obtained through rehydration after vitrification for a short period of time if necessary with PBS or the like and vitrify the sheet-shaped vitrigel again.

Examples of a drying method include various methods such as air drying, drying in a closed container (circulating air in a container and constantly supplying dry air), and drying in an environment in which silica gel is present. For example, as a method using air drying, a method for performing drying in an incubator kept sterile at 10° C. and 40% humidity for 2 days or performing drying in a clean bench in a sterile state all day and night at room temperature can be exemplified.

Also, when the sheet-shaped hydrogel dried body is irradiated with ultraviolet rays, it is possible to obtain a sheet-shaped vitrigel having excellent transparency and strength at the time of rehydration.

A method for preparing a sheet-shaped vitrigel dried body will be described below.

The method for preparing a sheet-shaped vitrigel dried body has a step C for obtaining a sheet-shaped vitrigel by rehydrating the sheet-shaped hydrogel dried body or the sheet-shaped hydrogel dried body irradiated with ultraviolet rays and a step D for obtaining a sheet-shaped vitrigel dried body by drying and revitrifying the sheet-shaped vitrigel.

[Step C]

Step C is a step for performing rehydration after vitrification (refer to (f) to (g) of FIG. 2). A sheet-shaped vitrigel is obtained through Step C.

Examples of the aqueous solution used for rehydration include sterilized water, physiological saline, PBS, and the like.

[Step D]

Step D is a step for obtaining a sheet-shaped vitrigel dried body by drying and revitrifying the sheet-shaped vitrigel. Furthermore, when the sheet-shaped vitrigel dried body is irradiated with ultraviolet rays, it is possible to increase a strength of the sheet-shaped vitrigel dried body (refer to (h) of FIG. 2).

An irradiation energy of the ultraviolet rays described above may be appropriately adjusted in accordance with a composition and content of the sheet-shaped hydrogel dried body or the sheet-shaped vitrigel dried body. The irradiation energy of the ultraviolet rays may be, for example, 0.1 mJ/cm² or more and 6000 mJ/cm² or less, for example, 10 mJ/cm² or more and 4000 mJ/cm² or less, and for example, 20 mJ/cm² or more and 500 mJ/cm² or less.

A thread is prepared using the sheet-shaped hydrogel dried body or the sheet-shaped vitrigel dried body prepared in this way. The sheet-shaped hydrogel dried body or the sheet-shaped vitrigel dried body may be hollowed out using a cylindrical blade or the like to form a through hole. When a thread is prepared using the sheet-shaped hydrogel dried body having such a through hole, it is possible to obtain a thread having fine pores. Examples of the cylindrical blade include a medical device such as a trepan. A shape of the blade may be appropriately changed in accordance with a cross-sectional shape of the through hole and is not limited to a cylindrical shape.

[Step E]

Step E is a step for obtaining a thread-shaped vitrigel by wetting the first sheet-shaped hydrogel dried body or the sheet-shaped vitrigel dried body in the first aqueous solution and twisting the same into a thread shape.

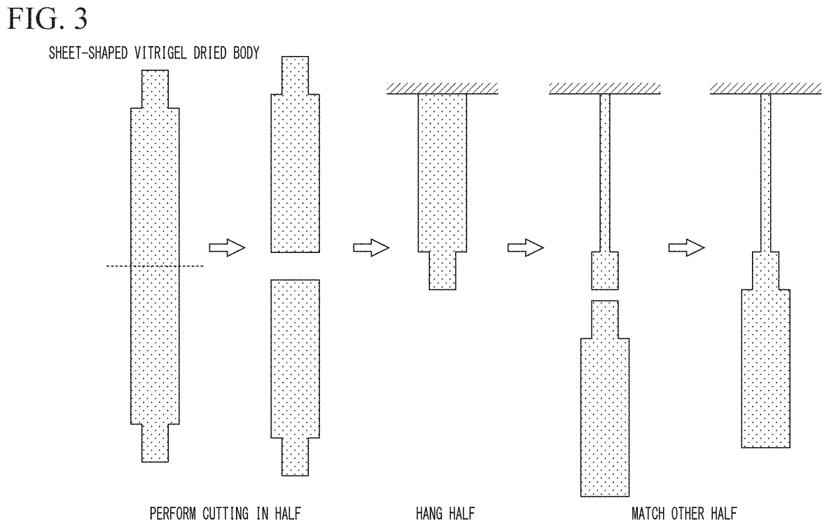

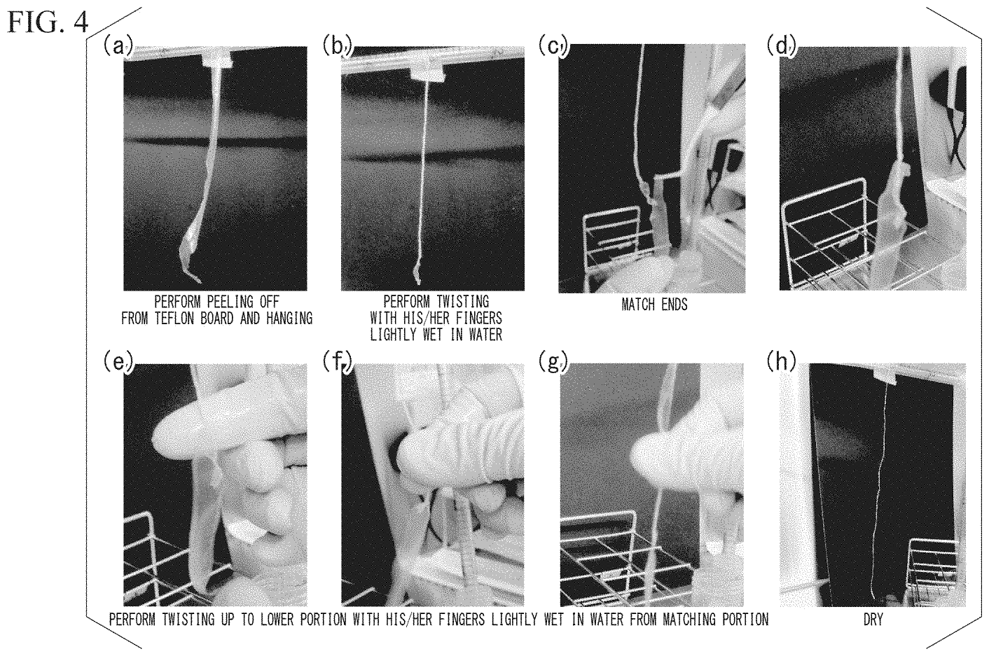

To be specific, as shown in FIG. 3, the obtained sheet-shaped hydrogel dried body or sheet-shaped vitrigel dried body is cut in half horizontally and one of the two halves is used as the first sheet-shaped hydrogel dried body or the sheet-shaped vitrigel dried body (the other is used as a second sheet-shaped hydrogel dried body or a sheet-shaped vitrigel dried body) in the Step F. First, an end portion of the first sheet-shaped hydrogel dried body or the sheet-shaped vitrigel dried body in which there is no protrusion portion is fixed to a support body such as a pipette. Subsequently, the fixed first sheet-shaped hydrogel dried body or sheet-shaped vitrigel dried body is twisted in the same direction while being moistened with the first aqueous solution to form a thread (refer to (a) and (b) of FIG. 4).

The first aqueous solution is not particularly limited, and examples thereof include sterilized water, physiological saline, PBS, and atelocollagen sol, and sterilized water and atelocollagen sol are preferable.

When it is desired to make the thread-shaped vitrigel longer, it is preferable that the preparation method in the embodiment has Step F.

[Step F]

Step F is a step for obtaining a thread-shaped vitrigel by overlapping an end portion, which is left without threading, of the first sheet-shaped hydrogel dried body or the sheet-shaped vitrigel dried body and an end portion of a second sheet-shaped hydrogel dried body or a sheet-shaped vitrigel dried body to form a joint portion and wetting the joint portion and the second sheet-shaped hydrogel dried body or the sheet-shaped vitrigel dried body in the first aqueous solution and twisting the same into a thread shape.

To be more specific, as shown in FIG. 3, a joint portion is formed by overlapping an end portion, which is left without threading, of the first sheet-shaped hydrogel dried body or the sheet-shaped vitrigel dried body and an end portion of a second sheet-shaped hydrogel dried body or a sheet-shaped vitrigel dried body (refer to (c) and (d) of FIG. 4). Subsequently, while performing wetting in the first aqueous solution, the wetting of a portion up to lower ends of the joint portion and the second sheet-shaped hydrogel dried body or the sheet-shaped vitrigel dried body is performed and the twisting of the same in the same direction is performed into a thread shape (refer to (e) to (g) of FIG. 4).

When Step F is repeatedly performed, it is possible to prepare a thread having an infinite length. Furthermore, the preparation method in the embodiment may have the following Steps G to 1.

[Step G]

Step G is a step for obtaining a thread-shaped vitrigel dried body by drying the thread-shaped vitrigel obtained in Step F (refer to (h) of FIG. 4).

[Step H]

Step H is a step for wetting the thread-shaped vitrigel dried body obtained in Step G in a second aqueous solution and then drying the same.

The second aqueous solution is not particularly limited, and examples thereof include sterilized water, physiological saline, PBS, and atelocollagen sol, and atelocollgen sol is preferable. When the thread-shaped vitrigel dried body is coated with atelocollagen sol, it is possible to increase a strength of a thread.

[Step I]

Step I is a step for irradiating the thread-shaped vitrigel dried body obtained in Step H or Step G with ultraviolet rays.

The strength of the ultraviolet rays is the same as that described in Step D. In order to perform uniform irradiation of the thread-shaped vitrigel dried body with ultraviolet rays, it is preferable to irradiate the thread-shaped vitrigel dried body with ultraviolet rays in a symmetrical direction a plurality of times.

When the thread-shaped vitrigel dried body is irradiated with ultraviolet rays, it is possible to increase a strength of a thread by forming a crosslinked structure in a molecule.

<Use>

The thread obtained through the preparation method in the embodiment can be used as, for example, a tissue regenerating thread, a carrier for cell transplantation, or the like. As described above, according to the embodiment, since a thread having an infinite length can be prepared, it is possible to perform knitting into a shape of an organ using the thread and it is possible to suitably use the same as a carrier for cell transplantation.

<<Thread>>

In the embodiment, the present invention provides a thread including a vitrigel dried body and having elasticity at the time of hydration. In the preparation method described above, when the twisting step is provided, the thread has elasticity. In addition, when the twisting step is provided, the thread has a spiral structure.

Examples of the sol used as a raw material for the vitrigel dried body include sols similar to the sols exemplified in the method for preparing a thread described above. Among them, as the vitrigel dried body constituting the thread in the embodiment, an atelocollagen vitrigel dried body is preferable because it is a biocompatible material.

### EXAMPLES

Although the present invention will be described below with reference to Examples, the present invention is not limited to the following Examples.

### [Preparation Example 1] Preparation of Thread

1. A mold having eight strips of 10 mm×200 mm (2.5 mm×20 mm cut at both ends: refer to FIG. 1) cut out in a PET film (thickness: 75 μm) with an A4 size was placed on a Teflon (registered trademark) board (refer to (a) of FIG. 2).

2.8 mL of a 1% atelocollagen solution was poured into 8 mL of a serum-free culture medium on ice and pipetting was performed three times to prepare uniform atelocollagen sol.

3.3.6 mL of atelocollagen sol was poured into each strip-shaped mold to spread throughout (four locations).

4. The above steps 2 and 3 were repeatedly performed and the atelocollagen sol was poured into a total of eight molds (refer to (b) of FIG. 2).

5. The mold injected with the atelocollagen sol was left for two hours in a 5% CO₂ incubator at 37° C. and gelled (refer to (c) of FIG. 2).

6. After gelation, the PET film was removed (refer to (d) of FIG. 2) and vitrified in a constant temperature and humidity chamber with a humidity of 40% and a temperature of 10° C. (refer to (e) of FIG. 2).

7. After vitrification, sterilized water was added, washed three times, and rehydrated (refer to (f) of FIG. 2). After rehydration (refer to (g) of FIG. 2), revitrification was performed in a constant temperature and humidity chamber with a humidity of 40% and a temperature of 10° C.

8. After revitrification, the atelocollagen vitrigel membrane dried body was irradiated with UV at 50 ml/cm² (refer to (h) of FIG. 2)

9. After UV irradiation, a strip-shaped atelocollagen vitrigel membrane dried body was peeled off from a Teflon (registered trademark) board, cut in half and attached to a pipette with one end prepared and hung it with a scotch mending tape (refer to FIG. 3 and (a) of FIG. 4).

10. The strip-shaped atelocollagen vitrigel membrane dried body was wetted with sterilized water and twisted in the same direction into a thread shape. A distal end of the strip (a 5 mm×20 mm portion) was left without threading (refer to (b) of FIG. 4).

11. A distal end (a 5 mm×20 mm portion) of another strip-shaped atelocollagen vitrigel membrane dried body and the remaining lower end overlapped (refer to FIG. 3 and (c) and (d) of FIG. 4), wetted with sterilized water, and twisted in the same direction. Subsequently, after a portion up to the lower end was wetted with sterilized water and twisted in the same direction (refer to (e) to (h) of FIG. 4), a clip was made into a heavy stone and dried.

12. The steps 9 to 11 described above were repeatedly performed to prepare four thread-shaped atelocollagen vitrigel dried bodies (twisting method: only sterilized water).

13. Two of the four prepared bodies were twisted in the same direction while being further wetted with atelocollagen sol and then the clip was made into a heavy stone and dried (twisting method: sterilized water+atelocollagen sol coating).

14. The steps 10 and 11 described above were newly performed using atelocollagen sol instead of sterilized water to prepare two thread-shaped atelocollagen vitrigel dried bodies (twisting method: atelocollagen sol).

15. Each body of the steps 12 to 14 was irradiated with UV at 400 mJ/cm² twice in a symmetrical direction.

### [Example 1] Strength Confirmation Test of Thread-Shaped Atelocollagen Vitrigel Dried Body

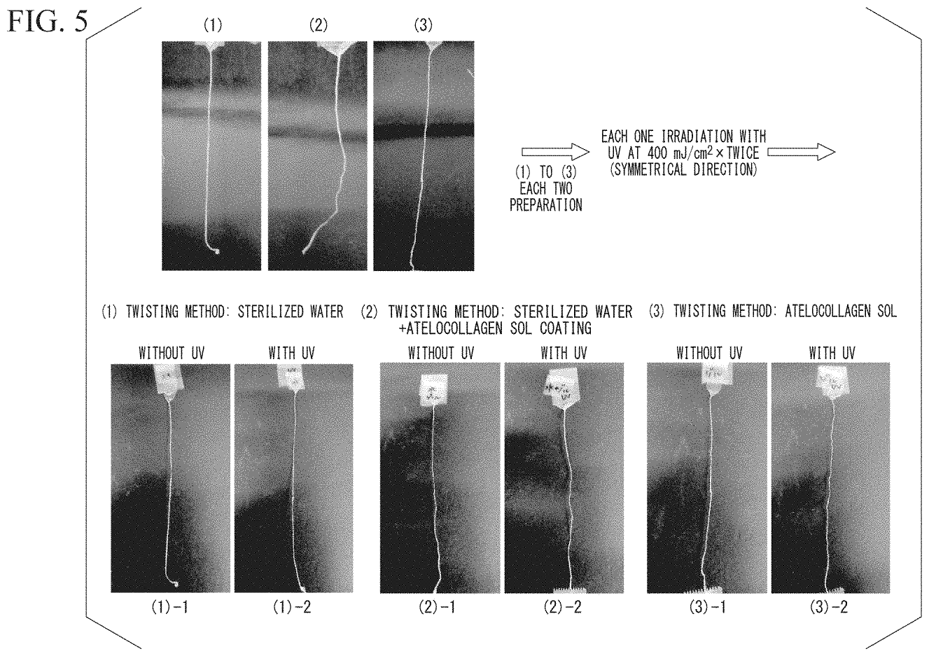

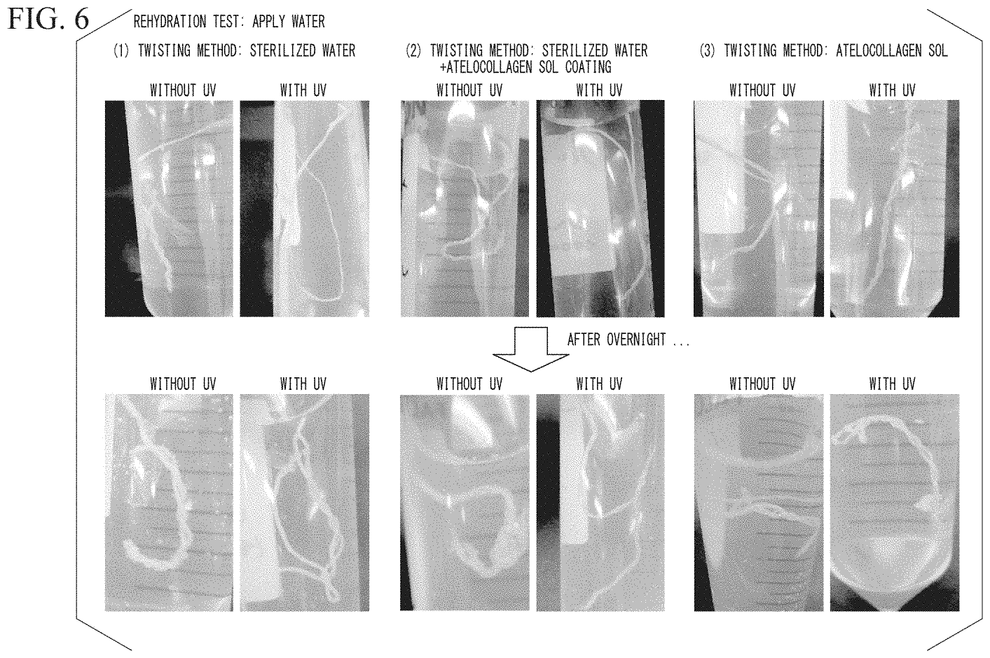

Each sample of the thread-shaped atelocollagen vitrigel dried body prepared in Preparation Example 1 was placed in a 50 mL conical tube including sterilized water and rehydrated. One day after rehydration, each sample was taken out and both ends of a thread were pulled to observe a degree of unraveling (refer to FIG. 6). The samples had the following six types: (1)-1: A thread (twisting method: sterilized water), (1)-2: a sample of a thread irradiated with UV (twisting method: sterilized water), (2)-1: a thread (twisting method: sterilized water+atelocollagen sol coating), (2)-2: a sample of a thread irradiated with UV (twisting method: sterilized water+atelocollagen sol coating), (3)-1: a thread (twisting method: atelocollagen sol), and (3)-2: a sample of a thread irradiated with UV (twisting method: atelocollagen sol) (refer to FIG. 5).

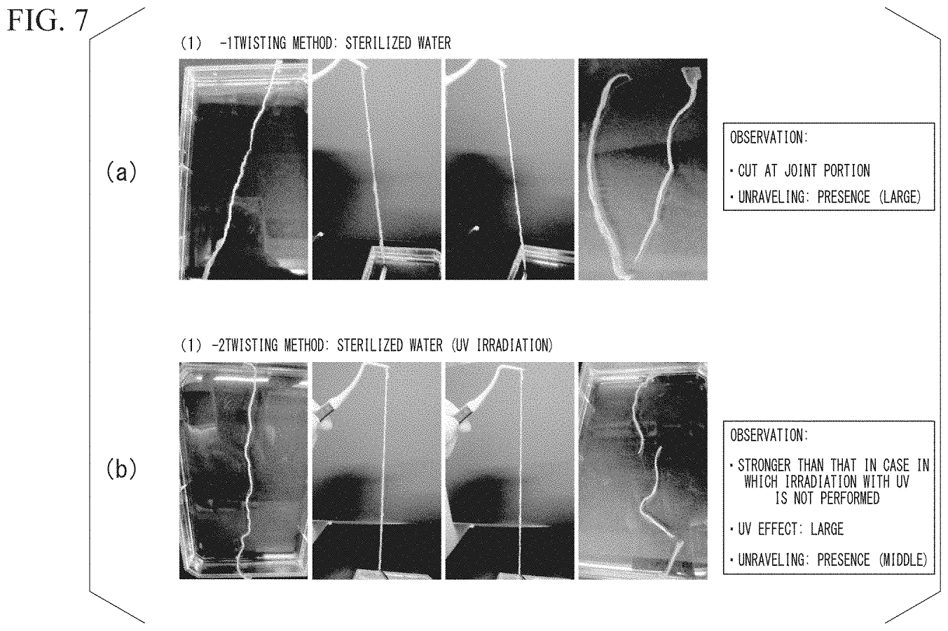

The results are shown in FIGS. 7 to 9.

As shown in (a) of FIG. 7, a thread prepared by performing twisting using sterilized water showed a large unraveling, and when both ends thereof were pulled, the thread was cut at a joint portion thereof. As shown in (b) of FIG. 7, a thread prepared by performing twisting using sterilized water and then performing irradiation with UV showed middle unraveling, and when both ends thereof were pulled, the thread was cut at a joint portion thereof, but a strength thereof was stronger than that of a thread which did not undergo irradiation with UV.

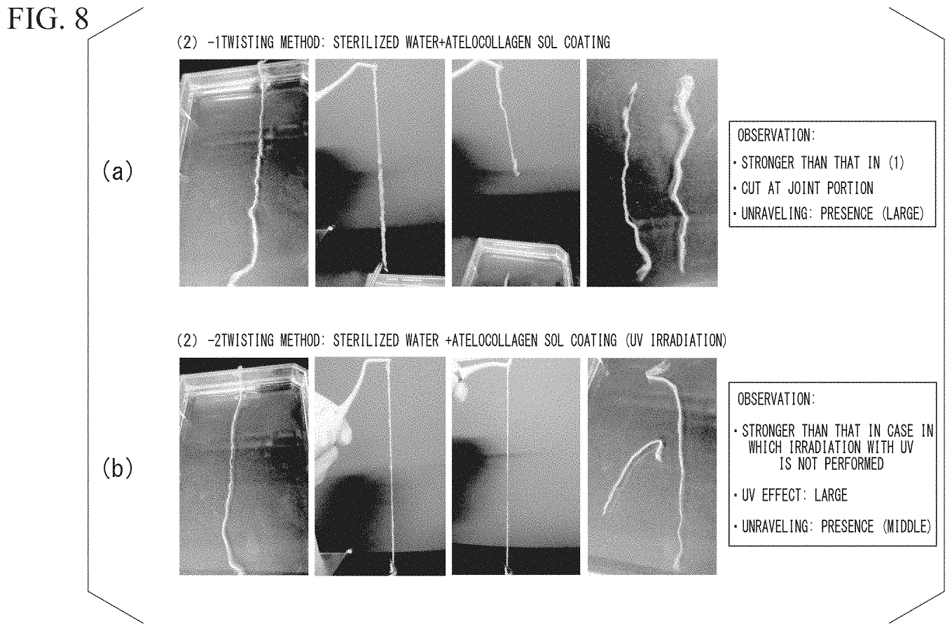

As shown in (a) of FIG. 8, a thread prepared by performing twisting using sterilized water and then performing atelocollagen sol coating showed a large unraveling, and when both ends thereof were pulled, the thread was cut at a joint portion thereof, but a strength thereof was stronger than that of a thread prepared only by performing twisting using sterilized water. As shown in (b) of FIG. 8, a thread prepared by performing twisting using sterilized water, performing atelocollagen sol coating, and then performing irradiation with UV showed middle unraveling, and when both ends thereof were pulled, the thread was cut at a joint portion thereof, but a strength thereof was stronger than that of a thread which did not undergo irradiation with UV.

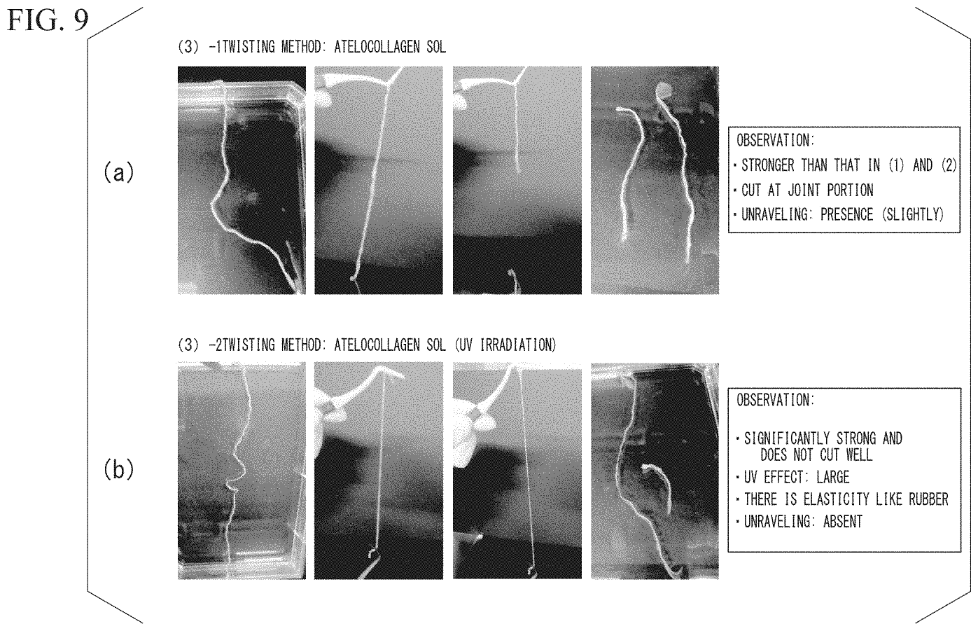

As shown in (a) of FIG. 9, a thread prepared by performing twisting in atelocollagen sol showed slight unraveling, and when both ends thereof were pulled, the thread was cut at a joint portion thereof, but a strength thereof was stronger than that of a thread prepared by performing twisting in sterilized water, a thread prepared by performing twisting in sterilized water and then performing irradiation with UV, a thread prepared by performing twisting in sterilized water and then performing atelocollagen sol coating, and a thread prepared by performing twisting in sterilized water, performing atelocollagen sol coating, and then performing irradiation with UV. As shown in (b) of FIG. 9, a thread prepared by performing twisting in atelocollagen sol and then performing irradiation with UV did not show unraveling, had elasticity like rubber, and was not easily cut even when both ends thereof were pulled, but a strength thereof was stronger than that of a thread which did not undergo irradiation with UV.

### INDUSTRIAL APPLICABILITY

According to the present invention, it is possible to provide an easy technique for preparing a thread-shaped atelocollagen vitrigel having an infinite length in which a strength thereof can be adjusted in accordance with an application and a dried body thereof.

1. A method for preparing a thread, comprising:
a step for obtaining a thread-shaped vitrigel by wetting a first sheet-shaped hydrogel dried body or a sheet-shaped vitrigel dried body in a first aqueous solution and twisting the same into a thread shape.

2. The method for preparing a thread according to claim 1, comprising:
a step for obtaining a thread-shaped vitrigel by overlapping an end portion, which is left without threading, of the first sheet-shaped hydrogel dried body or the sheet-shaped vitrigel dried body and an end portion of a second sheet-shaped hydrogel dried body or a sheet-shaped vitrigel dried body to form a joint portion and wetting the joint portion and the second sheet-shaped hydrogel dried body or the sheet-shaped vitrigel dried body in the first aqueous solution and twisting the same into a thread shape.

3. The method for preparing a thread according to claim 1, comprising:
a step for obtaining a thread-shaped vitrigel dried body by drying the thread-shaped vitrigel.

4. The method for preparing a thread according to claim 3, comprising:
a step for wetting the thread-shaped vitrigel dried body in a second aqueous solution and drying the same.

5. The method for preparing a thread according to claim 3, comprising:
a step for irradiating the thread-shaped vitrigel dried body with ultraviolet rays.

6. The method for preparing a thread according to claim 1, wherein the hydrogel is atelocollagen gel.

7. The method for preparing a thread according to claim 1, wherein the first aqueous solution is water or atelocollagen sol.

8. The method for preparing a thread according to claim 2, wherein the end portion is a protrusion portion.

9. The method for preparing a thread according to claim 4, wherein the second aqueous solution is atelocollagen sol.

10. The method for preparing a thread according to claim 1, comprising:
a step for obtaining a sheet-shaped hydrogel by injecting sol into a mold, gelling the sol, and then removing the mold; and
a step for obtaining a sheet-shaped hydrogel dried body by drying and vitrifying the sheet-shaped hydrogel.

11. The method for preparing a thread according to claim 10, comprising:
a step for irradiating the sheet-shaped hydrogel dried body with ultraviolet rays.

12. The method for preparing a thread according to claim 10, comprising:
a step for obtaining a sheet-shaped vitrigel by rehydrating the sheet-shaped hydrogel dried body or the sheet-shaped hydrogel dried body irradiated with ultraviolet rays; and
a step for obtaining a sheet-shaped vitrigel dried body by drying and revitrifying the sheet-shaped vitrigel.

13. The method for preparing a thread according to claim 12, comprising:
a step for irradiating the sheet-shaped vitrigel dried body with ultraviolet rays.

14. A thread, comprising:
a vitrigel dried body;
wherein the thread has elasticity at the time of hydration.

15. The thread according to claim 14, which has a spiral structure.

16. The thread according to claim 14, wherein the vitrigel dried body is an atelocollagen vitrigel dried body.
